(19) United States
(12) Patent Application Publication
(10) Pub. No.: US 2025/0319146 A1
(43) Pub. Date: Oct. 16, 2025

(54) Anti-Oxidant Repair Ganoderma Lucidum Extract and Preparation Method Therefor

(71) Applicant: Guangdong Yuewei Edible Fungi Technology Co., Ltd., Guangzhou, Guangdong (CN)

(72) Inventors: Jian Chen, Guangzhou (CN); Chunwei Jiao, Guangzhou (CN); Yizhen Xie, Guangzhou (CN); Qingping Wu, Guangzhou (CN); Jiaming Chen, Guangzhou (CN)

(21) Appl. No.: 18/692,974

(22) PCT Filed: Nov. 17, 2021
(86) PCT No.: PCT/CN2021/131083

§ 371 Date: Aug. 12, 2024

(30) Foreign Application Priority Data
Sep. 18, 2021 (CN) 202111097716.4

(51) Int. Cl. A61K 36/074; A23L 31/00; B01D 11/02; B01D 21/26; B01D 61/14; B01D 69/02; B01D 71/02

(52) U.S. Cl. CPC A61K 36/074; A23L 31/00; B01D 11/0288; B01D 21/262; B01D 61/147; B01D 69/02; B01D 71/02; A61K 2236/331; A61K 2236/37; A61K 2236/53; B01D 2325/02834

(57) ABSTRACT

The present invention relates to the technical field of preparation of food and cosmetic raw materials. Disclosed are an antioxidant repair Ganoderma lucidum extract and a preparation method therefor. The preparation method comprises: mixing crushed fruit body of Ganoderma lucidum with pure water, and heating and dynamically extracting to obtain a crude extract; standing the obtained crude extract, naturally settling, filtering a supernatant by using a screen mesh, and collecting a crude filtrate; using a disc stack centrifuge to perform secondary impurity removal on the obtained crude filtrate so as to obtain a filtrate; performing fine filtration by using a ceramic membrane having a pore diameter of 400-1200 nm, and collecting a permeate; and finally, concentrating and drying to obtain an antioxidant repair Ganoderma lucidum extract. The preparation method of the present invention is simple to operate and low in cost, and has the advantage of large scale. The finished product is high in controllability and stability, and the Ganoderma lucidum extract which is wide in application and good in effect can be provided for the industries of food, health food, and cosmetics.

### FIELD OF INVENTION

The invention relates to the technical field of preparation of food and cosmetics raw material, and particularly relates to an antioxidant repair Ganoderma lucidum extract and a preparation method thereof.

### TECHNICAL BACKGROUND

Ganoderma lucidum is a world-famous medicinal and edible fungus, which has been used as a medicinal tonic for more than 2,000 years in China. Because of its efficacy, Ganoderma lucidum has been referred to as “Ruicao”, “Linzhi Cao”, “Wannianrong” or “Xiancao” in China. It has the functions of regulating human immune function, prolonging life, anti-tumor and neuroprotection. At present, Ganoderma lucidum is listed as a legal crude drug in China Pharmacopoeia 2015, which has the function of anti-cancer, neurodegenerative diseases and cardiovascular diseases in clinic. In view of the good pharmacological effects of Ganoderma lucidum, natural active products extracted from Ganoderma lucidum fruit body or spores have been widely used in the research and development of food, health food and cosmetics. At present, there are many reports on the preparation methods of Ganoderma lucidum extract, and organic solvents are widely used in the preparation process, but it is difficult to remove impurities in the later stage. Or adopt a simple screen filtering mode of water extraction to remove mushroom residues, and that obtained extract is easy to precipitate after redissolution, which is extremely unstable, and is not suitable for being directly used for preparing Ganoderma lucidum drinks or cosmetics with high requirements on clarity, thus limiting its large-scale application. For example, (1) Patent CN1634537A discloses a Ganoderma lucidum extract, its preparation method and application. It discloses that Ganoderma lucidum extract is obtained with the Sporoderm-broken spore powder of Ganoderma lucidum as raw material, by being degreased with petroleum ether, extracted with ethanol under reflux, then re-extracted with chloroform and ethyl acetate, and finally recovered and dried under reduced pressure to get Ganoderma lucidum extract. Although the preparation method can obtain Ganoderma lucidum extract with excellent solubility, the steps are complicated with a large number of harmful solvents introduced and with high cost, which makes it difficult to have large scale production. (2) Patent CN104825500A discloses a Ganoderma leucocontextum extract, its extraction method and application, wherein one or more solvents of water, methanol, 95% (v/v) ethanol, ethyl acetate, dichloromethane or acetone are used for extraction, and then one of ethyl acetate, chloroform, dichloromethane and n-butanol is used for extraction to prepare the Ganoderma lucidum extract. This method can prepare an extract with good solubility, but it is difficult to remove impurities in the later stage because of the extraction with organic solvent. (3) Patent CN106924110A discloses the preparation and application of a Ganoderma lucidum extract, which is obtained by using water, ethanol or butanediol as extraction solvent and coarse filtering. This is a comparatively simple method with only simple filtering, which may result in a large number of insoluble substances in the prepared extract. (4) Patent CN112048024A discloses a Ganoderma lucidum extract, its preparation method and application. After extracting Ganoderma lucidum spore powder, centrifugal separation and micro-filtration, the retentate after micro-filtration is recovered, resulting in more insoluble substances in the Ganoderma lucidum extract. (5) Patent CN107050068A discloses an extraction method of Ganoderma lucidum extract and its application. The Ganoderma lucidum extract is obtained by using ethanol as the extraction solvent, and then roughly filtered with a screen. Ganoderma lucidum extract prepared by this method also contains a large number of tiny insoluble substances. (6) Patent CN107652371A discloses a preparation method of Ganoderma cornutum extract. It is mentioned that the equipment used in the preparation includes high-pressure pump, heat exchanger, reactor, sintering filter, cooler, sintering filter, minimum pressure valve and test tube. This preparation method is complicated with extremely high equipment requirement. In addition, patent CN113116758A discloses a preparation method of Ganoderma lucidum extract, in which the Ganoderma lucidum extract is placed at 2-8° C. for 20-40 days, a clarifying agent is added, and then filtered. Although Ganoderma lucidum extract with high clarity can be obtained by this method, the whole preparation process takes a lot of time.

In view of the shortcomings of the existing preparation method of Ganoderma lucidum extract, it is urgent to develop a Ganoderma lucidum extract which can be quickly and massively prepared and widely used in the research and development of various foods, health foods and cosmetics.

### SUMMARY

In view of the shortcomings and deficiencies in the prior art, the primary purpose of the invention is to provide a preparation method of an antioxidant repair Ganoderma lucidum extract. The simple preparation method does not need organic solvent extraction and other operations, and is suitable for large-scale production of Ganoderma lucidum extract, which provides Ganoderma lucidum extract with high activity, wide application and high polysaccharide content for the rapidly developing industries such as food, health food and cosmetics.

Another objective of the present invention is to provide an antioxidant repair Ganoderma lucidum extract prepared by the above method.

The purpose of the invention is realized by the following technical solution:

A preparation method of an antioxidant repair Ganoderma lucidum extract comprises the following preparation steps:
(1) Dynamic extraction with hot water: mixing the crushed fruit body of Ganoderma lucidum with pure water, heating, stirring and extracting to obtain a crude extract;
(2) Coarse filtration: the coarse extract obtained in step (1) is allowed to stand and naturally settle, the supernatant is taken and filtered by a screen with the rude filtration collected;
(3) Centrifugation: the crude filtration obtained in step (2) is subject to secondary impurity removal by using a disc stack centrifuge to obtain filtrate;
(4) micro-filtration: the filtrate obtained in step (3) is filtered by a ceramic membrane with the aperture of 400˜1200 nm, and the permeate is collected;
(5) concentrating and drying the permeate in step (4) to obtain the antioxidant repair Ganoderma lucidum extract.

Further, the mass ratio of the fruit body of Ganoderma lucidum mixed with pure water in step (1) is 1:20-1:40.

Further, the temperature of heating and stirring extraction in step (1) is 60˜95, with the stirring speed of 90˜180 rpm, and the extraction time 0.5˜3 h. Further preferably, the above-mentioned extraction time is 2 hours.

Further, the pore size of the screen in step (2) is 80-200 meshes.

Further, the filtered residue in step (2) is further extracted for multiple times by the methods in steps (1) and (2), and the crude filtrates are combined.

Further, the pore size of the ceramic membrane in step (4) is preferably 400˜800 nm.

Further, the residual retentate filtered by the ceramic membrane in step (4) is further diluted with pure water and then filtered repeatedly, and the permeate is combined.

Further, the concentration in step (5) is concentrated by vacuum concentration or reverse osmosis, so that the solid content in the concentrated solution is 80-200 g/L. More preferably, the solid content in the concentrated solution is 120-160 g/L.

Further, the drying in step (5) adopts vacuum drying or spray drying.

Furthermore, the content of polysaccharide in the antioxidant repair Ganoderma lucidum extract obtained in step (5) is 12-15% by mass.

An antioxidant repair Ganoderma lucidum extract is prepared by the above method.

Compared with the prior art, the invention has the following beneficial effects that:

(1) The preparation method of the invention adopts a hot water dynamic leaching process, which improves the extraction rate of active substances. There is no need to introduce organic solvent in the extraction process, which effectively reduces the pollution of organic solvent and the subsequent impurity removal operation.

(2) The preparation method of the invention adopts a large-aperture ceramic membrane to finely filter the water extract of Ganoderma lucidum, which not only ensures the clarity of the solution, but also removes large-particle inactive substances, and greatly reduces the interception of macromolecular active substances such as polysaccharides and proteins in the microfiltration process.

(3) The preparation method is simple in operation and low in cost, and is suitable for large-scale industrial production.

### BRIEF DESCRIPTION OF DRAWINGS

FIG. 1 is a standard curve graph of glucose obtained in Example 1;

FIG. 2 shows the results of polysaccharide content determination of Ganoderma lucidum extract obtained in Example 3 and Ganoderma leucocontextum extract obtained in Example 4;

FIG. 3 is a comparative diagram of DPPH free radical scavenging ability of Ganoderma lucidum extract and Ganoderma leucocontextum extract tested in Example 5;

FIG. 4 is a comparative diagram of ABTS free radical scavenging ability of Ganoderma lucidum extract and Ganoderma leucocontextum tested in example 6;

FIG. 5 is a comparative diagram of tyrosinase activity inhibition ability of Ganoderma lucidum extract and Ganoderma leucocontextum extract tested in Example 7.

### DETAILED DESCRIPTION

The present invention will be described in further detail with reference to examples and drawings, but the embodiments of the present invention are not limited thereto.

### Example 1

This example is the production of glucose standard curve graph:

(1) Accurately weigh 50 mg of analytically pure glucose (dried to constant weight at 105 g of analytically pure glucose (dried ents of the present invenmL, and obtain a glucose standard solution of 100 btamL.

(2) Suck 0.00 mL, 0.20 mL, 0.40 mL, 0.60 mL, 0.80 mL and 1.00 mL of the glucose standard solution into six colorimetric tubes, and add 1.00 mL, 0.80 mL, 0.60 mL, 0.40 mL, 0.20 mL and 0.00 mL of pure water in turn.

(3) Add 1.5 ml of 5% phenol solution and 7.0 mL of concentrated sulfuric acid into the above test tubes, shake well and react for half an hour. After cooling, the OD488 value was detected by spectrophotometer at 488 nm.

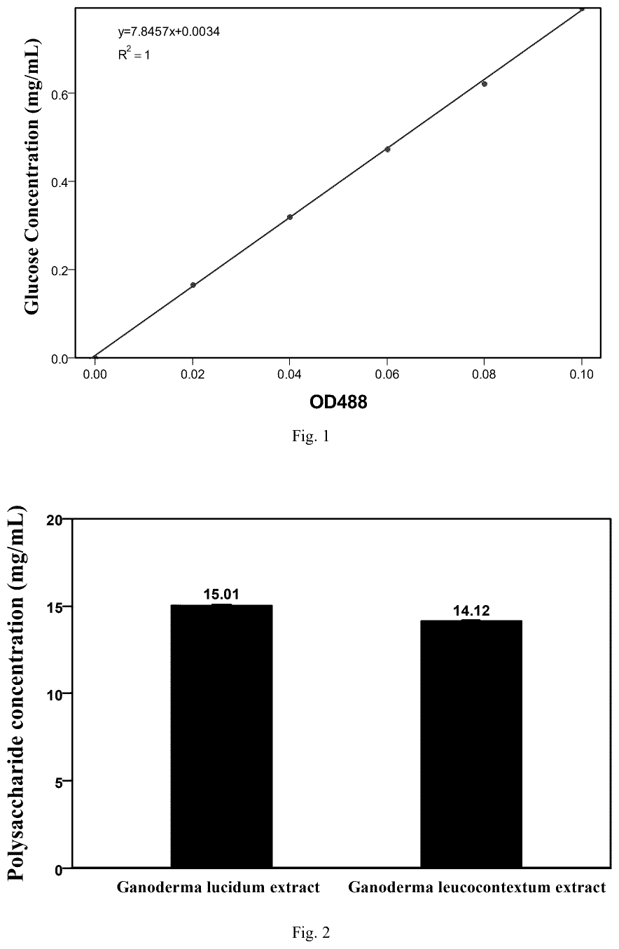

(4) Draw a glucose standard curve graph according to the ratio of concentration to absorbance, and the result is shown in FIG. 1.

### Example 2

This example is a method for determining the polysaccharide content of Ganoderma lucidum extract:

(1) Accurately weigh 1 g of Ganoderma lucidum extract and dissolve it in 100 mL of distilled water to prepare 1% (m/v) Ganoderma lucidum extract solution.

(2) Alcohol precipitation of polysaccharide: take 10 mL of the above 1% solution, add 50 mL of absolute ethanol, and precipitate overnight after shaking evenly.

(3) Centrifuge the polysaccharide alcohol precipitate in step (2) at 4000 rpm for 15 min, discard the supernatant, take the precipitate, add 30 mL of anhydrous ethanol, repeat the operation twice, and dissolve the precipitate with distilled water to a constant volume of 200 mL to prepare a polysaccharide solution.

(4) Absorb 1 mL of the polysaccharide solution in a 20 mL colorimetric tube, add 1.5 mL of phenol and 7.0 mL of concentrated sulfuric acid, shake well, and react for half an hour. After cooling, the OD488 value was detected by spectrophotometer at 488 nm.

(5) Calculate the total polysaccharide content in Ganoderma lucidum extract by using the glucose standard curve obtained in Example 1.

### Example 3

This example is the preparation of an antioxidant repair Ganoderma lucidum extract:

(1) Take 30 kilograms of crushed fruit body of Ganoderma lucidum, and add pure water according to the material-liquid ratio of 1:30 to prepare Ganoderma lucidum fruit body suspension. The suspension was heated to 95° C., stirred and extracted at 120 rpm for 2 hours to obtain a crude extract.

(2) The crude extract after the extraction in step (1) is allowed to stand and naturally settle, and when the extract is cooled to below 50ing ththe OD488 value was detected by spectrophotometer at 488 nm.m.o200-mesh screen to separate solid from liquid, and the crude filtrate of Ganoderma lucidum was collected.

(3) Carrying out secondary impurity removal on the crude Ganoderma lucidum filtrate in step (2) by using a disc stack centrifuge to obtain filtrate.

(4) Filtering the filtrate obtained in step (3) with a 800 nm ceramic membrane, and collecting the permeate; Dilute the retentate with 25 L pure water, re-filter, and combine the permeate.

(5) Then, the permeate obtained in the step (4) is concentrated by reverse osmosis to the Ganoderma lucidum aqueous extract concentrate with a solid content of about 120 g/L, and then spray drying is carried out to prepare the Ganoderma lucidum extract with antioxidant repair.

According to the method of Example 2, the polysaccharide content of Ganoderma lucidum extract obtained in this example was 15.01%. The corresponding determination results are shown in FIG. 2.

### Example 4

This example is the preparation of an antioxidant repair Ganoderma leucocontextum extract:

(1) Take 30 kilograms of crushed fruit body of Ganoderma leucocontextum, add pure water according to the ratio of material to liquid equal to 1:25, and make a Ganoderma leucocontextum fruit body suspension. Heating the suspension to 95° C., stirring at 90 rpm and extracting for 2 hours to obtain crude extract.

(2) The crude extract after the extraction in step (1) is allowed to stand and naturally settle, and when the extract is cooled to below 50e, the supernatant is extracted. Filtering the supernatant with a 200-mesh sieve to separate solid from liquid, and collecting the crude filtrate of Ganoderma leucocontextum.

(3) Performing secondary impurity removal on the Ganoderma leucocontextum coarse filtrate in step (2) by using a disc stack centrifuge to obtain a filtrate.

(4) Filtering the filtrate obtained in step (3) with a 800 nm ceramic membrane, and collecting the permeate. Dilute the retentate with 25 L pure water, re-filter, and combine the permeate.

(5) Subsequently, the permeate obtained in the step (4) is concentrated in vacuum to a Ganoderma leucocontextum water extraction concentrated solution with a solid content of about 140 g/L, and then spray drying is carried out to obtain the antioxidant repair Ganoderma leucocontextum extract. According to the method of Example 2, the polysaccharide content in the extract of Ganoderma leucocontextum extract.obtained in this example was 14.12%. The corresponding determination results are shown in FIG. 2.

### Example 5

In this example, the DPPH radical scavenging activity of the obtained Ganoderma lucidum extract was tested:

(1) Accurately weigh 0.0197 g DPPH, dissolve it in anhydrous ethanol and make it constant to 250 mL, and get 0.2 mmol/L DPPH working solution.

(2) Take a certain amount of Ganoderma lucidum extract of Example 3 or Ganoderma leucocontextum extract of Example 4 and use distilled water to prepare Ganoderma lucidum extract or Ganoderma leucocontextum extract solution with concentrations of 0, 0.1%, 0.2% and 0.5% respectively.

(3) Suck 50μ 3 of Ganoderma lucidum extract or Ganoderma leucocontextum extract solution with different concentrations and put them into 96-well plates, then add 150, of DPPH working solution, with three in each group in parallel. The reaction was conducted at room temperature in the dark for 1 hour. After the reaction was completed, the absorbance value (OD value) at 517 nm was measured by enzyme-labeled instrument.

(4) The measured DPPH clearance rate is calculated according to the following formula:

$$DDPH Scavenging Ability ( \% ) = \frac{{D}_{control} - ( {D}_{sample} - {D}_{blank} )}{{D}_{control}} \times 100 \%$$

In the formula, D_{control} is the OD value of DPPH solution without adding Ganoderma lucidum extract or Ganoderma leucocontextum extract solution, D_{blank} is the OD value of Ganoderma lucidum extract or Ganoderma leucocontextum extract solution itself, and Dₛₐₘₚₗₑ is the OD value of Ganoderma lucidum extract or Ganoderma leucocontextum extract solution after reacting with DPPH solution.

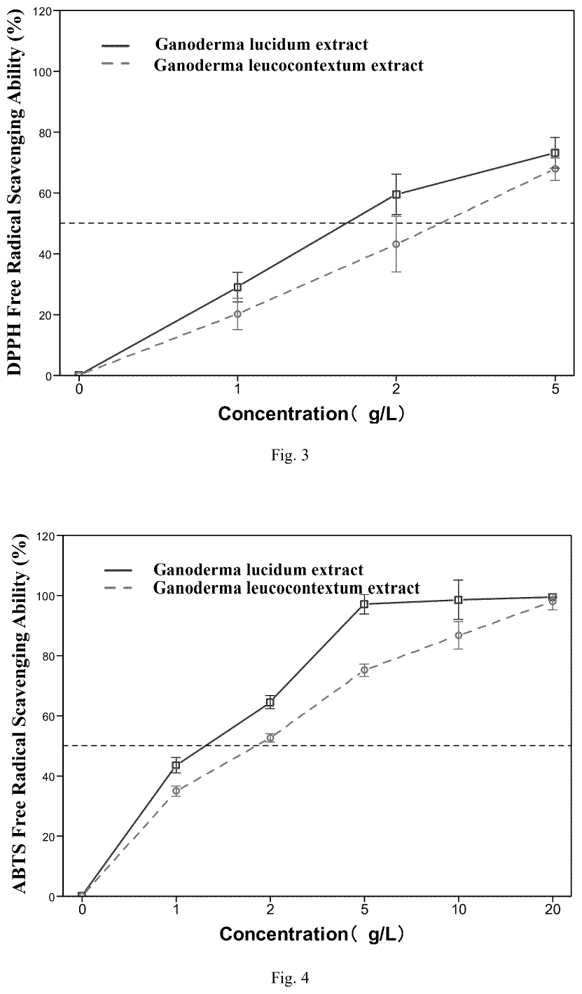

The test results are shown in FIG. 3. It can be seen from the results of FIG. 3 that the Ganoderma lucidum extract and the Ganoderma leucocontextum extract obtained by the invention have obvious DPPH free radical scavenging ability and show good antioxidant effect.

### Example 6

In this example, the ABTS free radical scavenging activity of the obtained Ganoderma lucidum extract was tested:

(1) Prepare 200 mL 0.2 mol/L phosphate buffer (PBS, pH 7.4).

(2) Take 17.57 mg of potassium persulfate and dissolve it in distilled water to a constant volume of 25 mL to prepare a 2.6 mM potassium persulfate solution.

(3) Dissolve 101.5 mg ABTS in distilled water, and make it to 25 mL to make 7.4 mM ABTS solution.

(4) Absorb 10 mL of potassium persulfate solution and ABTS solution respectively, mix them evenly in the same volume, avoid light and react at room temperature for 12-16 hours.

(5) Take a certain amount of Ganoderma lucidum extract of Example 3 or Ganoderma leucocontextum extract of Example 4 and use distilled water to prepare Ganoderma lucidum extract or Ganoderma leucocontextum extract solution with concentrations of 0, 0.1%, 0.2%, 0.5%, 1% and 2% respectively.

(6) Dilute the solution reacted in step (4) by 15 times with 0.2 mol/L phosphate buffer to prepare ABTS working solution for later use.

(7) Suck 50 the so Ganoderma lucidum extract or Ganoderma leucocontextum extract solution with different concentrations in step (5) and put them into 96-well plates, then add 150 king solution for later use.peratuturoom temperatuasured by enzyme-labeled instrument.eption of macromolecular active preparation intion innratisorbance at 734 nm was measured by enzyme-labeled instrument.

(8) The measured ABTS clearance rate is calculated according to the following formula:

$$ABTS Scavenging Ability ( \% ) = \frac{{D}_{control} - ( {D}_{sample} - {D}_{blank} )}{{D}_{control}} \times 100 \%$$

In the formula, D_{control} is the OD value of ABTS working solution without adding Ganoderma lucidum extract or Ganoderma leucocontextum extract solution, D_{blank} is the OD value of Ganoderma lucidum extract or Ganoderma leucocontextum extract solution itself, and Dₛₐₘₚₗₑ is the OD value of Ganoderma lucidum extract or Ganoderma leucocontextum extract solution after reacting with ABTS solution.

The test results are shown in FIG. 4. It can be seen from the results of FIG. 4 that the Ganoderma lucidum extract and the Ganoderma leucocontextum extract obtained by the invention have obvious ABTS free radical scavenging ability and show good antioxidant effect.

### Example 7

In this example, the tyrosinase inhibitory activity of the obtained Ganoderma lucidum extract was tested:

(1) Weigh 4.76 mg tyrosinase and dissolve it in water to 10 mL to make 476 6 tmL tyrosinase solution, then dilute 1 ml of 476 476 ktyrosinase solution to 5 ml to make 95.2 make 9tyrosinase working solution.

(2) Take a certain amount of Ganoderma lucidum extract of Example 3 or Ganoderma leucocontextum extract of Example 4 and use distilled water to prepare Ganoderma lucidum extract or Ganoderma leucocontextum extract solution with concentrations of 0, 0.1%, 0.2%, 0.5%, 1% and 2% respectively.

(3) Weigh 9.88 mg of LOPA and dissolve it in distilled water to a constant volume of 50 mL to prepare a 1 mM LOPA solution.

(4) Put 80 ion.n Ganoderma lucidum extract or Ganoderma leucocontextum extract solution in a 96-well enzyme-labeled plate, add 120 0 mL to mM DOPA solution, and finally add 50 n a 96tyrosinase working solution, with three in each group in parallel. The reaction was carried out at 37 out at 37ctively.espectively.for later use.peratuturoom temperatuasured by enzyme-labeled instrument.e

(5) The measured inhibition rate of tyrosinase activity is calculated according to the following formula:

$$Tyrosinase activity inhibition rate ( \% ) = \frac{{D}_{control} - ( {D}_{sample} - {D}_{blank} )}{{D}_{control}} \times 100 \%$$

In the formula, D_{control} is the OD value of DOPA solution and tyrosinase solution without adding Ganoderma lucidum extract or Ganoderma leucocontextum extract solution. D_{blank} is the OD value of Ganoderma lucidum extract or Ganoderma leucocontextum extract solution, and Dₛₐₘₚₗₑ is the OD value of Ganoderma lucidum extract or Ganoderma leucocontextum extract solution mixed with DOPA solution and tyrosinase solution.

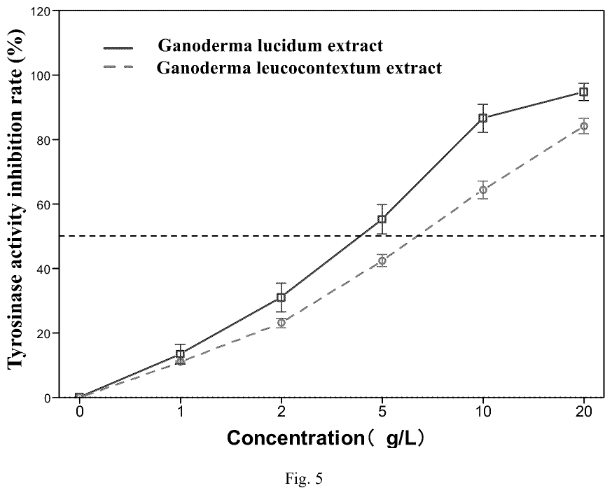

The test results are shown in FIG. 5. It can be seen from the results of FIG. 5 that both the Ganoderma lucidum extract and the Ganoderma leucocontextum extract obtained by the invention have significant tyrosinase inhibitory activity.

The above-mentioned embodiments are the preferred embodiments of the present invention, but the embodiments of the present invention are not limited by the above-mentioned embodiments, and any other changes, modifications, substitutions, combinations and simplifications made without departing from the spirit and principle of the present invention shall be equivalent substitutions, which are all included in the protection scope of the present invention.

1. A preparation method of an antioxidant repair Ganoderma lucidum extract, comprising the following preparation steps:
(1) Dynamic extraction with hot water: mixing crushed fruit body of Ganoderma lucidum with pure water, heating, stirring and extracting to obtain crude extract;
(2) Coarse filtration: the crude extract obtained in step (1) is allowed to stand and naturally settle. The supernatant is taken and filtered by a screen, and the crude filtrate is collected;
(3) Centrifugation: the crude filtrate obtained in step (2) is subject to secondary impurity removal by using a disc stack centrifuge to obtain filtrate;
(4) Microfiltration: the filtrate obtained in step (3) is filtered by a ceramic membrane with the aperture of 400 to 1200 nm, and the permeate is collected;
(5) Concentrating and drying the permeate in step (4) to obtain the antioxidant repair Ganoderma lucidum extract.

2. The preparation method of antioxidant repair Ganoderma lucidum extract according to claim 1, wherein the mass ratio of the fruit body of Ganoderma lucidum to pure water in step (1) is 1:20-1:40.

3. The preparation method of antioxidant repair Ganoderma lucidum extract according to claim 1, wherein the extraction temperature in step (1) is 60-95° C., the stirring speed is 90-180 rpm, and the extraction time is 0.5-3 hours.

4. The preparation method of antioxidant repair Ganoderma lucidum extract according to claim 2, wherein the pore size of the screen in step (2) is 80-200 meshes; the filtered residue is further extracted for many times by the methods of steps (1) and (2), and the crude filtrates are combined.

5. The preparation method of antioxidant repair Ganoderma lucidum extract according to claim 4, wherein the pore size of the ceramic membrane in step (4) is 400 to 800 nm.

6. The method for preparing the antioxidant repair Ganoderma lucidum extract according to claim 5, wherein the residual retentate filtered by the ceramic membrane in step (4) is further diluted with pure water, then filtered repeatedly, and the permeate is combined.

7. The preparation method of antioxidant repair Ganoderma lucidum extract according to claim 6, wherein the concentration in step (5) is concentrated by vacuum concentration or reverse osmosis, so that the solid content in the concentrated solution is 80-200 g/L.

8. The preparation method of antioxidant repair Ganoderma lucidum extract according to claim 6, wherein the drying in step (5) adopts vacuum drying or spray drying.

9. The preparation method of antioxidant repair Ganoderma lucidum extract according to claim 6, wherein the content of polysaccharide in the antioxidant repair Ganoderma lucidum extract obtained in step (5) is 12-15% by mass.

10. An antioxidant repair Ganoderma lucidum extract, prepared by the method according to claim 1.
